(19) United States
(12) Patent Application Publication
(10) Pub. No.: US 2016/0151543 A1
(43) Pub. Date: Jun. 2, 2016

(54) BIOCOMPATIBLE NANOPHASE MATERIALS

(71) Applicant: PURDUE RESEARCH FOUNDATION, West Lafayette, IN (US)

(72) Inventors: Thomas Jay WEBSTER, Barrington, RI (US); Karen Marie HABERSTROH, Barrington, RI (US); Saba CHOUDHARY, Bowie, MD (US)

(73) Assignee: PURDUE RESEARCH FOUNDATION, West Lafayette, IN (US)

(21) Appl. No.: 15/012,503

(22) Filed: Feb. 1, 2016

Related U.S. Application Data
(63) Continuation-in-part of application No. 12/067,082, filed on Mar. 17, 2008, now abandoned.
(60) Provisional application No. 60/718,623, filed on Sep. 20, 2005.

(51) Int. Cl. A61L 31/02; B22F 5/10; B22F 3/105; B22F 3/02; B22F 3/12

(52) U.S. Cl. CPC A61L 31/022; B22F 3/02; B22F 3/12; B22F 3/1055; B22F 5/106; B33Y 10/00

(57) ABSTRACT

Provided is an implantable composition having a metallic component, the metallic component being made of metal selected titanium, a titanium alloy, and CoCrMo, wherein the metal is formed from particles of nanophase powder having at least one dimension that is in the range of 1 to 2500 nm. Methods of making the implantable composition are also provided.

### CROSS-REFERENCE TO RELATED APPLICATIONS

This application is a continuation-in-part of U.S. National Phase Application No. 12/067,082, filed on Mar. 17, 2008, which was a U.S. national phase entry under §371 of International Application No.: PCT/US2006/036604, filed on Sep. 20, 2006, which claimed priority to U.S. Provisional Application No. 60/718,623, filed on Sep. 20, 2005, the entire contents of said applications being hereby incorporated herein by reference.

### BACKGROUND

A number of devices are being implanted in the body of animals, including humans. A large number of these devices include metallic components. The metals used to fabricate these components possess certain characteristics that result in the components having a medically acceptable degree of biocompatibility. For example, the metal component of an implanted device should possess appropriate properties so that it does not induce undesirable side effects. These undesirable side effects include blood clotting, tissue death, tumor formation, allergic reactions, foreign body reaction (rejection) and/or inflammatory reactions. Accordingly, it is desirable that these components integrate into a biological system to a medically acceptable degree and function as intended.

One example of a biological system exposed to the above discussed metallic components is the vascular system. For example, under certain circumstances, a metallic stent may be positioned within a lumen of a blood vessel. However, it should be understood that one or more stents can be positioned in the lumen of any body passageway if required (e.g., respiratory ducts, gastrointestinal ducts, urethra, esophagus and a bile duct and the like).

In one particular application stents are utilized to treat various vascular diseases. For example, atherosclerosis which is one of the leading causes of death in the world affecting approximately 58 million people. While there are many treatment options available for atherosclerosis (including angioplasty, orally prescribed pharmaceutical agents), when plaque build-up becomes severe, implantation of vascular stents into stenosed arteries is a desirable treatment option to help restore normal blood flow to ischemic organs.

In the past fifteen years, the use of stents has attracted an increasing amount of attention due the potential of these devices to be used as an alternative to surgery. Generally, a stent is used to obtain and maintain the patency of the body passageway while maintaining the integrity of the passageway. In one example, stents are useful in the treatment and repair of blood vessels after a stenosis has been compressed by percutaneous transluminal coronary angioplasty (PTCA), percutaneous transluminal angioplasty (PTA), or removed by atherectomy or other means, to help improve the results of the procedure and reduce the possibility of restenosis. Stents are also used to provide primary compression to a stenosis in cases in which no initial PTCA or PTA procedure is performed.

Accordingly, it is desirable to enhance the biocompatibility of metallic components implanted into the body of an animal.

### SUMMARY

An arrangement having a metallic component for implanting into the body of an animal in accordance with the present disclosure comprises one or more of the following features or combinations thereof. In addition a method for fabricating a metallic component for implanting into the body of an animal in accordance with the present disclosure comprises one or more of the following features or combinations thereof:

In one embodiment an arrangement for implanting in a body of an animal, comprises, a biocompatible metallic component having a nanophase surface. For example the surface has a number of structures thereon. The structures may be defined by a set of dimensions where at least one dimension of the set may be equal to or less than about 100 nm. The metallic component may include titanium. The metallic component may include CoCrMo. The metallic component may be, or include, a stent having a nanophase surface.

In another embodiment, a method of making a metallic biocompatable component may comprise compressing a nanophase metallic powder into a compact such that the compact has a nanophase surface. Particles of the nanophase metallic powder may have at least one dimension that is in the range of about 2500 nm to about 1 nm. For example, the nanophase metallic powder may be, or include, a titanium powder or alloy thereof, where a substantial number of the titanium powder particles may have at least one dimension that is equal to or about 2400 nm. In another example, the nanophase metallic powder may be, or include titanium powder where a substantial number of the titanium powder particles have at least one dimension that is equal to or about 500 nm. In another example, the nanophase metallic powder may be, or include, titanium powder where a substantial number of the titanium powder particles have at least one dimension that is equal to or about 750 nm. In another example, the nanophase metallic powder may be, or include, titanium powder where a substantial number of the titanium powder particles have at least one dimension that is equal to or about 250 nm. In another example, the nanophase metallic powder may be, or include, a titanium alloy may have at least one dimension that is equal to or about 1400 nm. In another embodiment, the particles of the nanophase powder may have at least one dimension that is in the range of about 2400 nm to about 200 nm. In another embodiment, the particles of the nanophase powder may have at least one dimension that is in the range of about 2000 nm to about 400 nm. In another embodiment, the particles of the nanophase powder may have at least one dimension that is in the range of about 1500 nm to about 600 nm. In another embodiment, the particles of the nanophase powder may have at least one dimension that is in the range of about 1000 nm to about 1 nm. In another embodiment, the particles of the nanophase powder may have at least one dimension that is in the range of about 2400 nm to about 500 nm. In another embodiment the particles of the nanophase powder may have at least one dimension that is in the range of about 400 nm to about 200 nm. In another embodiment, the particles of the nanophase powder may have at least one dimension that is in the range of about 100 nm to about 1 nm. In another embodiment, the particles of the nanophase powder may have at least one dimension that is in the range of about 750 nm to about 250 nm. In another embodiment, the particles of the nanophase powder may have at least one dimension that is less than or equal to about 500 nm. In another embodiment, the particles of the nanophase powder may have at least one dimension that is less than or equal to about 400 nm. In another embodiment, the particles of the nanophase powder may have at least one dimension that is less than or equal to about 300 nm. In another embodiment, the particles of the nanophase powder may have at least one dimension that is less than or equal to about 200 nm. In another embodiment, the particles of the nanophase powder may have at least one dimension that is less than or equal to about 100 nm. Furthermore, nanophase metallic powder may be, or include, CoCrMo powder where a substantial number of the CoCrMo powder particles may have at least one dimension that is equal to or about 400 nm. The nanophase metallic powder may be, or include, CoCrMo powder where a substantial number of the CoCrMo powder particles may have at least one dimension that is equal to or about 200 nm. The nanophase metallic powder may be, or include, CoCrMo powder where a substantial number of the CoCrMo powder particles may have at least one dimension that is in the range of about 400 nm to about 200 nm.

Additional features of the present disclosure will become apparent to those skilled in the art upon consideration of the following detailed description of preferred embodiments exemplifying the best mode of carrying out the subject matter of the disclosure as presently perceived.

### BRIEF DESCRIPTION OF THE DRAWINGS

FIG. 1 is a table showing the metal particle sizes as determined by AFM;

FIG. 2 is a table showing the surface roughness of metal compacts as determined by AFM;

FIG. 3A-FIG. 3D show scanning electron microscopy images of titanium compacts;

FIG. 4A-FIG. 4B show scanning election micrograph images of CoCrMo compacts;

FIG. 5A and 5B show scanning electron micrograph images of titanium particles;

FIG. 6 is a graph illustrating the increased RAEC adhesion oil nanophase titanium;

FIG. 7A and FIG. 7B show fluorescence microscopy images of enhanced spread morphology of live RAEC on nanophase titanium and conventional titanium surfaces;

FIG. 8 is a graph illustrating the increased RASMC adhesion on nanophase titanium;

FIG. 9A and FIG. 9B show fluorescence microscopy images of live RASMC on nanophase titanium and conventional titanium surfaces;

FIG. 10 is a graph illustrating the increased RAEC adhesion on nanophase and conventional CoCrMo surfaces.

FIG. 11 is a graph illustrating the increased RASMC adhesion on nanophase CoCrMo;

FIG. 12 shows fluorescence microscopy images of live RAEC grown on substrates on day 1, day 3, and day 5;

FIG. 13 shows fluorescence microscopy images of the RAEC remnants present on substrates after cell lysis;

FIG. 14 is a graph illustrating the increased RAEC growth on nanophase titanium;

FIG. 15 shows fluorescence microscopy images of live RASMC grown on substrates on day 1, day 3, and day 5;

FIG. 16 is a graph illustrating the increased RASMC growth on nanophase titanium;

FIG. 17 is a graph illustrating the collagen synthesis per RAEC on substrates;

FIG. 18 is a graph illustrating the collagen synthesis per RASMC on substrates;

FIG. 19 is a graph illustrating the elastin synthesis per RAEC on substrates;

FIG. 20 is a graph illustrating the elastin synthesis per RASMC on substrates; and

FIG. 21 shows a stent.

### DESCRIPTION

While the disclosure is susceptible to various modifications and alternative forms, specific embodiments will herein be described in detail. It should be understood, however, that there is no intent to limit the disclosure to the particular forms described, but on the contrary, the intention is to cover all modifications, equivalents, and alternatives falling within the spirit and scope of the disclosure.

The present disclosure generally relates to a metallic substance for implanting into the body of an animal. Note that an animal includes humans. The metallic substance may be configured as a component of an arrangement for implanting into a body of an animal. In addition, the metallic substance may be configured as the device for implantation into the body of an animal. The present disclosure also relates to methods for making such metallic substances.

A metallic substance of the present disclosure will possess characteristics which allow it to be implanted into the body of an animal. Metallic substances of the present disclosure will possess mechanical and chemical properties in order to function and exist in contact with the biological tissue of an animal, e.g., soft tissue. For example, the substance will possess the appropriate properties so it does not induce medically unacceptable reactions in the body such as blood clotting, tissue death, tumor formation, allergic reaction, foreign body reaction (rejection), and/or inflammatory reaction. In addition, the metallic substance will posses the appropriate strength, elasticity, permeability, and flexibility in order for it to function properly for its intended purpose. Moreover, it is desirable that the substance (i) sterilize easily and (ii) substantially maintain its physical properties during the time it remains in contact with biological tissue.

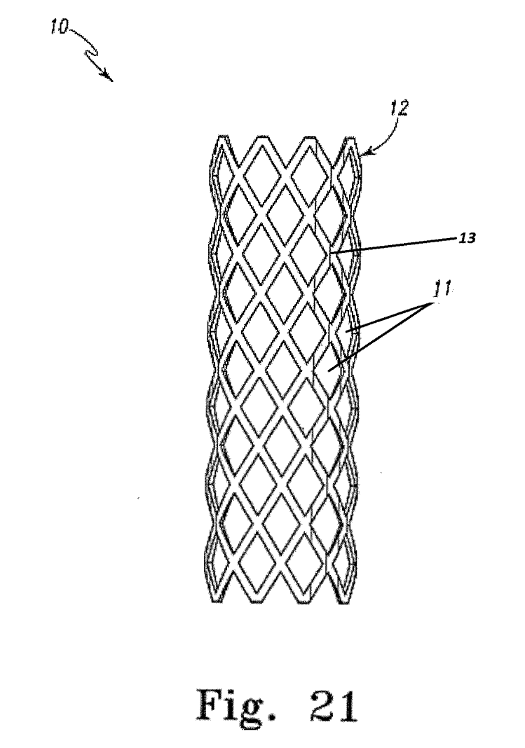

In one particular embodiment, a metallic substance of the present disclosure may be implanted in a passageway defined by soft tissue; for example a blood vessel. It should be appreciated that the metallic substance of the present disclosure may be positioned in the lumen of any soft tissue passageway in the body of an animal, such as respiratory ducts, gastrointestinal ducts, urethra, esophagus, bile ducts and the like. In one embodiment the metallic substance may be utilized in treating a vascular system of an animal such as blood vessels, such as arteries. In one embodiment the metallic substance may be configured as a stent, as shown in FIG. 21.

The metallic substances of the present disclosure may be fabricated from nanophase powder. Nanophase powder may be a powder composed of particles where a substantial number of particles have at least one dimension that is less than or equal to about 2500 nm. For example, a substantial number of the particles may have at least one dimension in the range of about 2400 nm to about 1 nm, or from about 2400 nm to about 200 nm, or from about 2000 nm to about 400 nm, or from about 1500 nm to about 600 nm, or from about 1000 nm to about 1 nm, or from about 2400 nm to about 500 nm, or from about 400 nm to about 200 nm, or from about 100 nm to about 1 nm. Furthermore, a substantial number of the particles may have at least one dimension less than or equal to about 500 nm, or less than or equal to about 400 nm, or less than or equal to about 300 nm, or less than or equal to about 200 nm, or less than or equal to about 100 nm.

Metallic substances of the present disclosure may have a nanophase surface. For example, the surface may have structures thereon where a substantial number of the surface structures have at least one dimension that is less than or equal to about 2500 nm. For example, a substantial number of the structures may have at least one dimension in the range of about 2400 nm to about 1 nm, or from about 2400 nm to about 200 nm, or from about 2000 nm to about 400 nm, or from about 1500 nm to about 600 nm, or from about 1000 nm to about 1 nm, or from about 2400 nm to about 500 nm, or from about 400 nm to about 200 nm, or from about 100 nm to about 1 nm. In addition, a substantial number of the structures may have at least one dimension less than or equal to about 500 nm, or less than or equal to about 400 nm, or less than or equal to about 300 nm, or less than or equal to about 200 nm, or less than or equal to about 100 nm.

In some embodiments, the inventive implantable composition comprises a metallic component, said metallic component consisting of metal selected from the group consisting of titanium, a titanium alloy, and CoCrMo, wherein the metal is formed from particles of nanophase powder having at least one dimension that is in the range of 1 to 2500 nm.

In some embodiments, the invention provides a method of making an implantable composition. For example, in some embodiments, the method comprises forming an implantable composition consisting of a metallic component (e.g., a metallic component consisting of a metal selected from the group consisting of titanium, a titanium alloy, and CoCrMo), wherein said forming comprises providing the metal in the form of particles of nanophase powder, and compacting the particles, thereby forming the implantable composition, wherein the particles of nanophase powder have at least one dimension that is in the range of 1 to 2500 nm.

In some embodiments, compacting the particles comprises sintering them. Sintering is a process of compacting and forming a mass of material by heat and/or pressure, for example, without melting it to the point of liquefaction. While sintering may optionally melt metal, various embodiments comprise sintering without melting the metal to the point of liquefaction. In some embodiments, a laser is used to compact the particles. For example, some embodiments, employ Selective Laser Sintering (SLS), which uses a laser as a power source to sinter the metal particles. In some embodiments of SLS, a laser is aimed automatically at points in space defined by a 3D model, binding metal particles together to create a solid structure.

In some embodiments, the implantable composition has a surface defined by a plurality of metallic structures, wherein said metallic structures are formed by particles of nanophase powder present at an exterior surface of the implantable composition, the metallic structures being defined by a set of dimensions (e.g., their width or height) wherein at least one dimension of the set is equal to or less than 500 nm.

In some embodiments of the implantable composition, a plurality of nanometer-sized voids having a diameter of less than 1 μm are distributed throughout the metallic component of the implantable composition. In such embodiments, the voids are interparticulate voids that are present by nature of the various metal particles interfacing with one another. In some embodiments, the interparticulate voids are present throughout the whole of the metallic component. In some embodiments, the interparticulate voids are homogenously (i.e., 90-100% uniformly) dispersed throughout the metallic component.

In some embodiments, the nanometer-sized voids have an average diameter between 10 and 1,000 nm (e.g., 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, or 990 nm), including any and all ranges and subranges therein.

In some embodiments, a plurality of holes (which may also be referred to as openings) are formed in the metallic component. For example, FIG. 21 depicts a stent 10, which is an implantable composition consisting of metallic component 13, wherein a plurality of diamond-shaped holes/openings 11 are present in the metallic component 13. The holes may be of any desired shape and size (e.g., round, ovular, triangular, diamond, etc.). In some embodiments, the holes/openings 11 have an average diameter of less than or equal to 1 mm. As used with respect to the holes/openings 11, a “diameter” refers to the longest of possible straight lines that may be drawn passing from side to side through the center of a body. For example, in FIG. 21, the diameter of the holes/openings 11 is determined by taking the vertical measurement of the length from the top of one of the diamond-shaped holes 11 to the bottom of the hole.

In some embodiments, the holes have an average diameter of 10 to 1,000 μm (e.g., 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330,340, 350, 360, 370, 380, 390, 400, 410, 420, 430, 440, 450, 460, 470, 480, 490, 500, 510, 520, 530, 540, 550, 560, 570, 580, 590, 600, 610, 620,630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nm), including any and all ranges and subranges there (e.g., 100 to 800 μm, 300 to 700 μm, etc.).

Methods of forming holes in compositions are well known, and, where present, holes may be formed in the inventive implantable compositions via any art-acceptable means. For example, holes may be present in a die or other 3-D mold or plan that may be used to make the implantable composition, or they may be added (e.g., via drilling or other means) after an intermediary implantable composition is formed.

### Substrates

Examples of materials which may be used to make the metallic substances of the present disclosure include commercially pure titanium (c.p. Ti), Ti6Al4V ELI, and Co28Cr6Mo. Powders were obtained from Powder Tech Associates (Bedford, Mass.). Nanophase and conventional particle sizes in each respective metal category (titanium, Ti6Al4V, and CoCrMo) were obtained. Each respective group of nanophase and conventional particulates possessed the same material properties (chemistry and shape) and altered only in dimension. Powders were loaded into a steel-tool die to obtain compacts. It should be appreciated that these compacts can be utilized in a process to fabricate various metallic components of a device for implantation into the body of an animal. In addition, these compacts can be utilized to fabricate the device itself, for example the stent 10 shown in FIG. 21 which has a nanophase surface 12. These compacts were used in the in cell experiments discussed below. In one application one pressure level (10 GPa over 5 min) was used to press all titanium-based compacts to green densities 90-95% of theoretical. At a different pressure level (5 GPa over 5 min), particles of the CoCr-based elemental blends were pressed. All pressed green discs (diameter: 12 mm, thickness: 0.50-1.10 mm) were produced using a simple uniaxial, single ended compacting hydraulic press (Carver, Inc). Powders were pressed in air at room temperature. Rolled, heat-treated, and pickled c.p. titanium sheets (wrought titanium; Osteonics) were used as controls during the cell experiments. Borosilicate glass (Fisher) etched in 10 N NaOH for 1 h was also utilized as a reference substrate in the cell experiments. All substrates were sterilized by first rinsing in ethanol, followed by ultraviolet (UV) light exposure for 2 h on each side.

In another embodiment, nanophase powders (˜1 g) were loaded into a steel-tool die and pressed under 4000 psi for titanium substrates and 5000 psi for CoCrMo substrates, each for 5 min. These compacts were pressed in air at room temperature using a uniaxial, single-ended compacting hydraulic press (Carver, Inc). In experiments with titanium, wrought titanium (Alfa Aesar) was used as a control and the tissue culture plate alone (Corning), which was made of polystyrene, was used as a reference substrate. In experiments with CoCrMo, borosilicate glass coverslips (Fisher) etched in 1 N NaOH for 1 h were used as preference substrate. All substrates were sterilized by first rinsing in ethanol, followed by ultraviolet (UV) light exposure for 2 h on each side.

The powders were characterized using scanning electron microscopy (SEM) and atomic force microscopy (AFM). It should be appreciated that with respect to titanium, an example of conventional particle size is greater than or about 10,500 nm. It should be appreciated that with respect to Ti6Al4V, an example of conventional particle size is greater than or about 7,500 nm. It should be appreciated that with respect to CoCrMo, an example of conventional particle size is in the range of, or about 44,000 nm to about 106,000 nm.

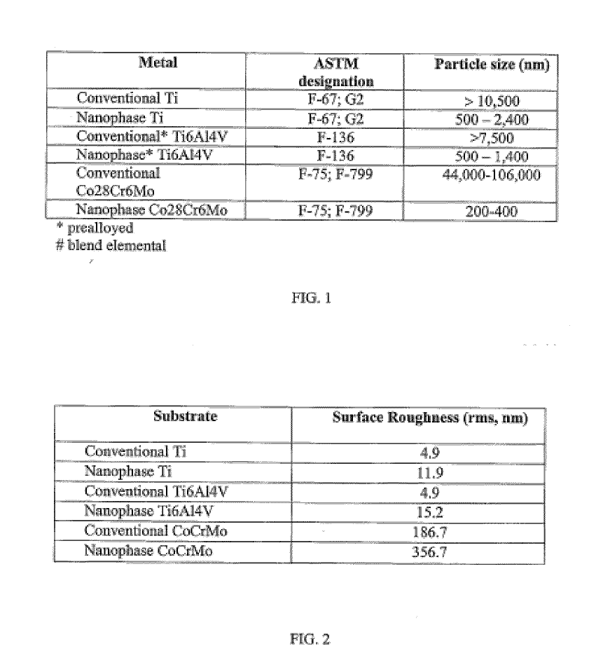

A table of particle size as determined by AFM, as shown in FIG. 1, illustrates the significant difference in particle sizes of nanophase and conventional metal powders. In particular, the nanophase titanium particles have a particle size in the range of about 500 nm to about 2,400 nm. In addition, the nanophase CoCrMo particles have a particle size in the range of about 400 nm to about 200 nm. Also, the nanophase Ti6Al4V particles have a particle size in the range of about 500 nm to about 1,400 nm.

A table of surface roughness of metal compacts as determined by AFM is shown in FIG. 2. The root mean square (rms) surface roughness values of nanophase and conventional metal compacts are shown. In particular, nanophase titanium compact has a surface roughness (rms) of 11.9 nm, which is about 2.5 times that of conventional titanium compact. In addition, nanophase Ti6Al4V compact has a surface roughness (rms) of 15.2 nm, which is about 3.1 times that of conventional titanium compact. Also, nanophase CoCrMo compact has a surface roughness (rms) of 356.7 nm, which is about 1.9 times that of conventional titanium compact.

### Cell Culture—Rat Aortic Endothelial Cells (RAEC)

The ability of the above described metallic substances to support cell proliferation and adhesion was determined as follows. Rat aortic endothelial cells (RAEC) were obtained from VEC Technologies (Rensselaer, N.Y.) and cultured in MCDB-131 Complete Medium (VEC Technologies). Cells were grown under standard cell culture conditions (i.e., a sterile, humidified, 95% air, 5% CO₂, 37° C. environment) on tissue culture polystyrene petri dishes (Corning) after being coated with a 0.2% gelatin (Sigma) solution in dH₂O.

RAEC were passaged after being cultured to confluence. Briefly, the existing media was aspirated and the cells were rinsed with 4 mL of phosphate-buffered saline (PBS; a solution containing 0.8% NaCl, 0.02% KCl, 0.15% Na₂HPO₄, and 0.02% KH₂PO₄ in dH₂O at a pH of 7.4; all chemicals were obtained from Sigma) and detached by rinsing with 1-2 mL of a trypsin/EDTA solution (containing 0.015% trypsin and 0.03% EDTA in a Hank's Balanced Salt Solution (0.01% MgCl₂, 0.01% MgSO₄, 0.04% KCl, 0.006% KH₂PO₄, 0.8% NaCl, 0.035% NaHCO₃, 0.009% Na₂HPO₄, and 0.1% d-glucose); all chemicals were obtained from Sigma). After passaging, the cells were transferred to new petri dishes coated with 0.2% gelatin, resuspended in fresh MCDB-131 Complete Medium, and further cultured under standard cell culture conditions. RAEC were used in experiments at population numbers ≦10 without further characterization.

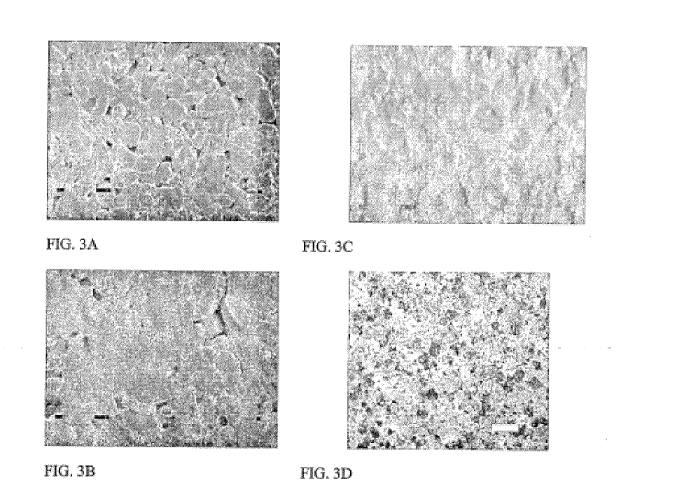

It should be understood that scanning electron micrographs of nanophase and conventional substrates show different surface topographies. Micrographs were taken using a JEOL JSM-840 Scanning Electron Microscope (Peabody, Mass.) at 3 kV with JEOL digital acquisition software. Specifically, FIG. 3A-FIG. 3D show scanning electron micrograph images of titanium compacts. Increased nanostructured surface toughness was observed in scanning electron microscopy images of nanostructured (FIG. 3A, Bar=10 micron) compared to conventional titanium (FIG. 3B, Bar=10 micron) and wrought titanium (FIG. 3C, Bar=1 micron). In contrast to nanostructured titanium compact surfaces, the optical microscopy image of wrought titanium surfaces (FIG. 3D, Bar=50 micron) acid etched to reveal grain size, indicated a large degree of microsurface roughness.

### Cell Culture—Rat Aortic Smooth Muscle Cells (RASMC)

Rat aortic smooth muscle cells (RASMC) were obtained from VEC Technologies (Rensselaer, N.Y.) and cultured in Dulbcecco's Modified Eagle's Medium (DMEM; Hyclone) supplemented with 10% fetal bovine serum (FBS; Hyclone) and 1% penicillin/streptomycin (P/S; Hyclone) under standard cell culture conditions directly on tissue culture polystyrene petri dishes.

RASMC were passaged after being cultured to confluence. Briefly, the existing media was aspirated and the cells were rinsed with 4 mL of PBS and detached from the petri dish with 1-2 mL of the trypsin/EDTA solution (prepared as described previously in section on RAEC preparation). After passaging, the cells were transferred to new petri dishes, resuspended in fresh DMEM supplemented with 10% FBS and 1% P/S, and further cultured under standard cell culture conditions. RASMC were used in experiments at population numbers ≦10 without further characterization.

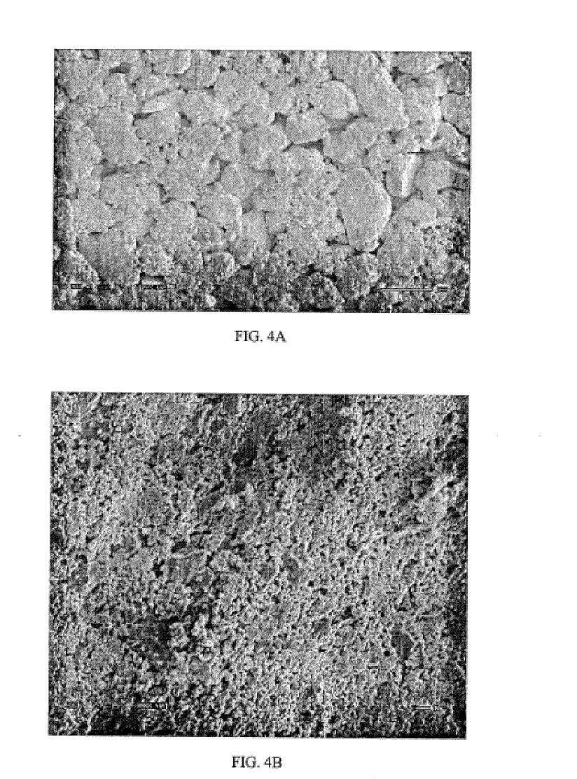

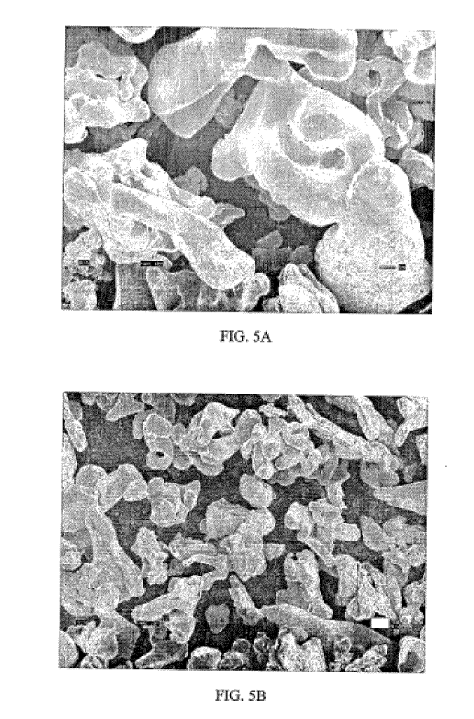

FIG. 4A and FIG. 4B show scanning electron micrograph images of CoCrMo surfaces (Scale bar=10 micron) for conventional CoCrMo (FIG. 4A) and for nanophase CoCrMo (FIG. 4B) surfaces. It should be noted that the nanophase surface has increased nanostructured surface roughness as shown in the table of FIG. 2.

The data demonstrates increased nanometer surface roughness in nanophase compared to conventional titanium, Ti6Al4V, and CoCrMo (see FIG. 3A-FIG. 3D, and FIG. 4A-FIG. 4B). The dimensions of nanometer surface features gave rise to larger amounts of interparticulate voids (with fairly homogeneous distribution) in nanophase titanium and Ti6Al4V, unlike the corresponding conventional titanium and Ti6Al4V compacts; these latter compacts revealed less interparticulate voids with a non-homogeneous distribution.

Spherical (Co) and irregular (Cr and Mo) powder particle elemental blends were pressed into nanophase CoCrMo (made from nanometer particle sizes: 200-400 nm) and into conventional CoCrMo (made from large micron particle sizes: 44,000-106,000 nm), as shown in the table in FIG. 1. Unlike conventional CoCrMo compacts, high interparticulate void density (number of voids per unit area) and nanometer void sizes (less than 1 mm) were exhibited on nanophase CoCrMo (FIG. 4A and FIG. 4B). Few relatively large particles can be seen with cleavage-like facets in nanophase CoCrMo. The substrates made out of coarse particles (conventional CoCrMo), in contrast, appeared only minimally deformed. The deformed particle size is within the 50-160 mm range. Interparticulate voids were large (10-50 mm) and void density was small for the conventional CoCrMo compacts. The exposed topography of the wrought titanium sheet (FIG. 3C and FIG. 3D) showed surface features in the range 20-60 mm. Moreover, after etching in an acidic (HF+HNO₂) aqueous solution, wrought titanium showed grain sizes in the traditional range of 20-50 mm (roughly equivalent to ASTM No. 7.5) under optical microscopy (FIG. 3D).

As discussed above the substrate made from nanoparticles exhibited nanostructured surface features and thus has a nanophase surface, whereas the substrate made from conventional particles has a conventional surface. Specifically, compacting these nanophase and conventional particles resulted in 3.1, 2.4, and 1.9 times more nanometer surface roughness on nanophase titanium alloy (Ti6Al4V), titanium and CoCrMo as compared to conventional titanium alloy (Ti6Al4V), titanium, and CoCrMo substrates, respectively (see FIG. 2). Due to this increase in surface roughness, increased surface area was also measured for the nanophase metallic surfaces as compared to conventional metallic surfaces. Specifically, 23%, 15% and 11% more surface area was measured on nanophase compacts compared to conventional titanium alloy (Ti6Al4V), titanium and CoCrMo compacts, respectively.

### Cell Proliferation, Adhesion, and Extracellular Matrix Production

For all experiments, substrates were placed in triplicate into the wells of a 12-well plate (Corning). For experiments with titanium, nanophase titanium, conventional titanium, and wrought titanium (control) were placed into the wells and the tissue culture plate alone (polystyrene) was used as a reference. For experiments with CoCrMo, nanophase CoCrMo, conventional CoCrMo, and etched glass coverslips (reference) were placed into the wells.

To each well of the plates, 2 mL of fresh media (either MCDB-131 Complete Medium for RAEC or DMEM supplemented with 10% FBS and 1% P/S for RASMC) was added. Next, cells that were grown to confluence were rinsed with PBS, detached with the trypsin/EDTA solution (prepared as described previously in section on RAEC preparation), resuspended in media, and counted with a hemocytometer.

For adhesion experiments with titanium substrates, RAEC or RASMC were seeded at a density of 3,500 cells/cm2 into each well and were allowed to adhere onto the substrate for 4 h under standard cell culture conditions. Adhesion experiments with CoCrMo substrates were carried out in the same way except that RAEC or RASMC were seeded at 7,000 cells/cm2. For proliferation and extracellular matrix production experiments, RAEC or RASMC were seeded at 50,000 cells per well and allowed to adhere for 4 h. After this time, the substrates were rinsed with PBS to remove non-adherent cells, the medium was changed, and the cells were allowed to grow for 1, 3, and 5 days. The medium was changed on days 1 and 3.

### Direct Cell Counts

At each of the prescribed time points, the substrates were washed with PBS to remove non-adherent cells and adherent cells were stained and viewed under a Leica DM IRB fluorescence microscope (McHenry, Ill.) as described below.

For experiments with Ti substrates, adherent cells were stained using a live/dead assay (Molecular Probes). Briefly, the live stain contains calcein AM and the dead stain contains ethidium homodimer-1. Live cells were distinguished by the presence of ubiquitous intracellular esterase activity that converted the nonfluorescent cell-permeant calcein AM to the intensely fluorescent calcein. The calcein dye produced an intense green fluorescence in live cells after being excited with blue light. Dead cells were distinguished after ethidium homodimer-1 entered damaged cell membranes and underwent a 40-fold enhancement in fluorescence after binding to nucleic acids. This produced an intense red fluorescence in dead cells after being excited with green light. Live (green) and dead (red) cells were counted under a fluorescence microscope; images were taken using a digital camera (Hamamatsu ORCA-ER) and ImagePro Plus 4.5 software.

For experiments with CoCrMo substrates, adherent cells were fixed with formaldehyde and stained with Hoechst 33258 (Molecular Probes). This dye emitted blue fluorescence when bound to double-stranded DNA. The stained nuclei (blue) were counted under a fluorescence microscope.

For all experiments, cells in at least five random fields were counted for each substrate, averaged, and divided by the area of the field of view to obtain the cell density (cells/cm²). All experiments were run in triplicate and repeated at least three independent times.

### Cell Lysate Collection

Following RAEC or RASMC proliferation experiments, cell lysate samples were collected for use in the CytoTox96 Cell Count, Sircol Collagen, and Fastin Elastin assays (each assay has been described in detail in the sections that follow). Importantly, cells used to collect lysate samples were not previously stained.

For cell lysis, substrates were transferred to fresh 12-well plates. Next, 1 mL of lysis solution (1% Triton X-100 in PBS; Triton X-100 was obtained from Sigma) was added to each well and the plates were incubated in 37° C. for 45 min. After incubation, the lysis solution in each well was mixed thoroughly by pipetting up and down numerous times. The cells were further lysed in a freeze/thaw cycle by placing the plates in −80° C. for 30 min to freeze and then quickly transferring them to 37° C. to thaw. The lysis solution in each well was again thoroughly mixed by pipetting up and down numerous times. The final lysed cell solution in each well was transferred to its respective labeled microcentrifuge tube end centrifuged at 250×g for 4 min. The supernatant was collected as the cell lysate.

### Cell Count Assay

The cell lysates collected from 1,3, and 5 day cell proliferation experiments were used in the CytoTox96® Non-Radioactive Cytotoxicity Assay (Promega) in order to obtain a cell count of each lysate sample. This assay measured the amount of lactate dehydrogenase (LDH) that released upon cell lysis. An enzymatic reaction between the released LDH and assay components converted tetrazolium salt into a red formazan product. The amount of red color formed was measured using a plate reader at an absorbance of 490 nm and was directly proportional to the number of cells lysed.

For this assay, 50 μL of cell lysate (collected as described previously) was added in triplicate to the wells of a 96-well plate (Corning). To each well containing lysate, 50 μl of reconstituted Substrate Mix (Promega) was added; the plate was then wrapped in aluminum foil to protect it from light and allowed to incubate at room temperature for 30 min. After incubation, 50 μl of Stop Solution (Promega) was added to each well. All bubbles were burst using a syringe needle. The SpectraMAX 190 plate reader (Molecular Devices Corp.) and SOFTmax Pro 3.1.2 software (Molecular Devices Corp.) was used to measure absorbance at 490 nm.

Each cell count assay was perforated with standards of 1,562; 3,125; 6,250; 12,500; 25,000; 50,000; and 100,000 cells/mL. A standard curve was created by plotting the known cell count values against the corresponding absorbance values at 490 nm. The equation of the standard curve was used to determine the cell count of the unknown samples. All cell count assays were run in triplicate and repeated at least three independent times.

### Collagen Quantification

A commercially available kit, Sircol™ Collagen Assay (Biocolor), was used to quantify the amount of collagen produced by RAEC or RASMC after 3 and 5 days. This assay is a quantitative dye-binding assay that utilized Sirius Red, which is a dye that has specific affinity for collagen under the specified assay conditions. The assay is capable of measuring mammalian collagens (Types I to V).

For the assay, 200 μl of each cell lysate sample was placed into duplicate microcentrifuge tubes. One mL of Sircol Dye Reagent (Biocolor) was added to each tube and mixed by inverting. The tubes were then placed on an orbital shaker for 30 min to allow for the Sircol Dye to bind to soluble collagens and precipitate out of solution. The tubes were then centrifuged at 10,000×g for 10 minutes to pack the collagen-dye complex. The supernatant (unbound dye solution) was discarded by decanting. To release the bound dye, 1 mL of Alkali Reagent (Biocolor) was added to each tube and the tubes were vortexed to dislodge the collagen-dye pellet. The tubes were placed on an orbital shaker for 10 min to allow the released dye to dissolve and mix. From each tube, 200 μl of the solution was placed into a well of a 96-well plate. All bubbles were burst using a syringe needle. The SpectraMAX 190 plate reader (Molecular Devices Corp.) and SOFTmax Pro 3.1.2 software (Molecular Devices Corp.) was used to measure absorbance values at 540 nm.

Each collagen assay was performed with standards of 2.5, 5, 10, 12.5, 25, and 50 μg collagen per 200 μl. A standard curve was created by plotting the known collagen concentration values against the corresponding absorbance values at 540 nm. The equation of the standard curve was used to determine the collagen concentration of the unknown samples. To obtain collagen production per cell, the overall collagen production was divided by the cell count number. All collagen assays were ran in duplicate and repeated at least three independent times.

### Elastin Quantification

A commercially available kit, Fastin™ Elastin Assay (Biocolor), was used to quantify the amount of elastin produced by RAEC or RASMC after 3 and 5 days. This assay is capable of measuring mammalian elastins through a quantitative dye-binding method. The assay utilized 5,10,15,20-tetraphenyl-21,23-porphrine sulphonate (TPPS), which is a dye that has affinity for elastin under the specified assay conditions.

For the assay, 100 μl of each cell lysate sample was placed into duplicate microcentrifuge tubes. One mL of cold Elastin Precipitating Reagent (pre-cooled to 4° C.; Biocolor) was added to each tube and mixed by inverting. All tubes were placed on ice and set in the refrigerator overnight. The following morning, the old ice was replaced with fresh ice and the tubes were returned to the refrigerator for an additional 30 min. While still ice cold, the tubes were centrifuged at 10,000×g for 10 min to pack the precipitated elastin. The supernatant was discarded by decanting. To each tube, 200 μl of 90% Saturated Ammonium Sulfate (Biocolor) and 1 mL of Fastin Dye Reagent (Biocolor) were added. Each tube was vortexed to dislodge the elastin pellet. The tubes were placed on an orbital shaker for 1 h to allow the dye to interact with the elastin, forming an elastin -dye complex, which became insoluble in the presence of the ammonium sulfate and precipitated out of solution. The tubes were then centrifuged at 10,000×g for 10 min to pack the elastin-dye complex. The supernatant (unbound dye solution) was discarded by decanting. To release the bound dye, 1 mL of Fastin Dissociation Reagent (Biocolor) was added to each tube and the tubes were vortexed to dislodge the elastin-dye pellet. The tubes were placed on an orbital shaker for 10 min to allow the released dye to dissolve and mix. From each tube, 100 μL of the solution was placed into a well of a 96-well plate. All bubbles were burst using a syringe needle. The SpectraMAX 190 plate reader (Molecular Devices Corp.) and SOFTmax Pro 3.1.2 software (Molecular Devices Corp.) was used to measure absorbance at 405 nm.

Each elastin assay was performed with standards of 5, 10, 12.5, 25, 50, 75 μg elastin per 100 μL. A standard curve was created by plotting the known elastin concentration values against the corresponding absorbance values at 405 nm. The equation of the standard curve was used to determine the elastin concentration of the unknown samples. To obtain elastin production per cell, the overall elastin production was divided by the cell count number. All elastin assays were run in duplicate and repeated at least three independent times.

### Statistics

All data are expressed as mean values±standard error of the mean (SEM). Two-tailed student t-tests were used to assess statistically significant differences between experimental data values. Resulting p values that were less than 0.1 were considered statistically significant.

### Titanium Substrate Characterization

FIG. 5A and FIG. 5B show scanning electron micrograph images of the difference in particle size for two different gram sized (conventional and nanophase) titanium particles (scale bar=10 micron) before they were compacted into substrate. It can be observed that the nanoparticles smaller than the conventional particles from which the nanophase and conventional substrates were made, respectively. Scanning electron micrographs depicting the two different surface topographies of the compacted substrates are shown in FIG. 3A-FIG. 3D. These substrates also exhibit the differences in grain size; the surface of the nanophase titanium substrate possesses much smaller grains than its conventional counterpart. These substrates were used to evaluate the responses of endothelial and vascular smooth muscle cells to metals with different surface topographies (i.e., nanophase and conventional).

### RAEC and RASMC Adhesion on Ti Substances

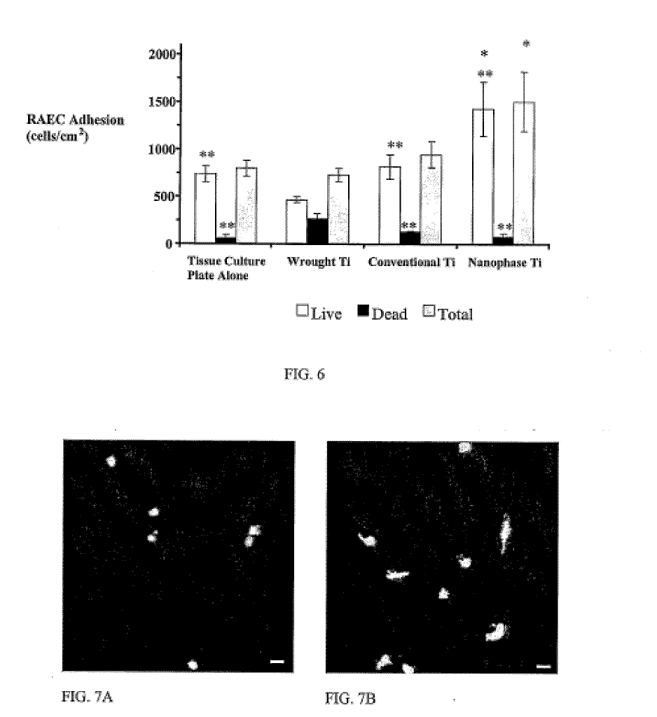

The adhesion response of endothelial cells was tested on nanophase and conventional Ti substrates. FIG. 6 is a graph which depicts the number of live, dead, and total Rat Aortic Endothelial Cells (RAEC) found adherent to each substrate after the 4 h adhesion period. The graph in FIG. 6 shows the increased Rat Aortic Endothelial Cells (RAEC) adhesion on nanophase titanium compact versus, wrought titanium, conventional titanium, and control (tissue culture plate alone) (data presented are mean values±SEM; n=3; *p<0.05 compared to respective conventional titanium sample, **p<0.05 compared to respective wrought titanium sample). Results show a statistically significant increase (p<0.05) in the number of live and total endothelial cells adherent to nanophase titanium as compared to conventional titanium and wrought titanium. There was no significant difference between the total number of endothelial cells adherent to conventional titanium and wrought titanium; however, there was a statistically significant increase (p<0.05) in the number of live cells adherent to conventional titanium as compared to wrought titanium.

In addition, the majority of endothelial cells adherent to the substrates were live, with the exception of a few dead cells. The number of dead cells found adherent to nanophase titanium was not significantly different from those found on the tissue culture plate alone, suggesting that nanophase titanium had no effect on the viability of endothelial cells; this also holds true when comparing dead endothelial cells on conventional titanium and the tissue culture plate alone. However, wrought titanium yielded a significantly higher (p<0.05) number of dead cells as compared to all other substrates tested.

Finally, fluorescence microscopy images confirmed this enhanced endothelial cell adhesion on nanophase as compared to conventional Ti. This preference of endothelial cells was further depleted by a more well-spread morphology on nanophase Ti, in contrast to a more “ball-shaped” morphology on conventional Ti as shown by fluorescence microscopy images (20× magnification) in FIG. 7A and FIG. 7B.

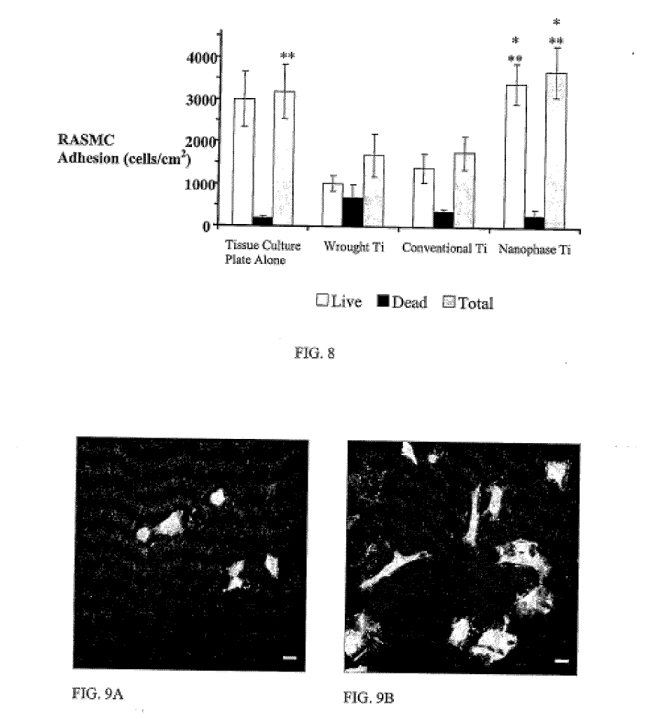

Vascular smooth muscle cells responded in a similar manner when grown on Ti substrates. Namely, FIG. 8 depicts the number of live, dead, and total vascular smooth muscle cells found adherent to each substrate after the 4 h adhesion period (Data are mean values±SEM; n=3; *p<0.01 compared to respective conventional titanium sample, **p<0.01 compared to respective wrought titanium sample). As was observed for endothelial cells, a significantly higher (p<0.01) density of live and total vascular smooth muscle cells were found adherent to nanophase titanium as compared to conventional titanium and wrought titanium.

In addition, the majority of adherent vascular smooth muscle cells were live, with the exception of a small number of dead cells found on all substrates. The number of dead cells adherent on the titanium substrates was not significantly different from the number of dead cells adherent on the tissue culture plate alone, suggesting that titanium had no effect on the viability of vascular smooth muscle cells.

Finally, fluorescence microscopy images (20× magnification) confirmed this enhanced vascular smooth muscle cell adhesion on nanophase titanium as compared to conventional titanium. This preference of smooth muscle cells was further depicted by a more well-spread morphology on nanophase titanium, in contrast to a more “ball-shaped” morphology on conventional titanium, as shown in FIG. 9A and FIG. 9B (scale bare=20 micron).

### RAEC and RASMC Adhesion on CoCrMo Alloy Substrates

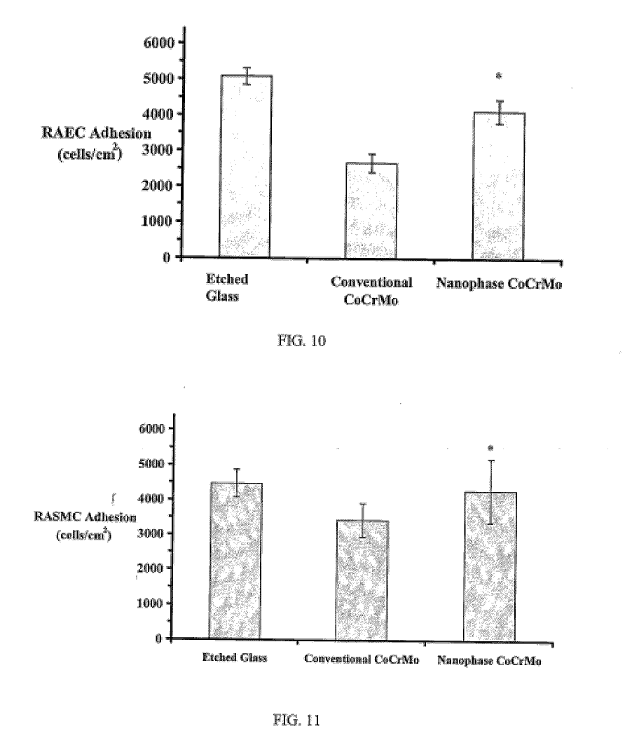

In order to evaluate whether the preference of RAEC and RASMC to nanophase titanium was material specific, another metal (namely, a CoCrMo alloy) was tested. Specifically, adhesion experiments were carried out using nanophase and conventional CoCrMo substrates. FIG. 10 depicts the number of total endothelial cells adherent on each substrate (data are mean values±SEM; n=3; *p−0.01 compared to conventional CoCrMo). Results show a statistically significant increase (p<0.01) in the number of endothelial cells adherent on nanophase CoCrMo as compared to conventional CoCrMo.

It should be appreciated that titanium and CoCrMo alloy particles in both nanophase and conventional regime were obtained, compacted, and used without further chemical or heat treatments in the present disclosure. This method of substrate preparation eliminated other surface variables such as those introduced by heat treatments and chemical etching methods. For example, previous studies confirm that using NaOH treatment for creating nanostructured features on the surface of PLGA decreased endothelial cell adhesion and proliferation (as compared to conventional PLGA); tins was due in part to chemical alterations of the etching process, since a casting method used to create the nanostructured topography (in the absence of chemical changes) on PLGA substrates increased endothelial cell adhesion and proliferation. Accordingly, such confounding variables were avoided in older to attribute any changes in endothelial and vascular smooth muscle cell adhesion and other functions to the nanostructured surface features of the metallic substances.

Vascular smooth muscle cells responded in a similar manner when allowed to adhere to CoCrMo substrates as shown in FIG. 11 (data are mean values±SEM; n=3; *p<0.1 compared to conventional CoCrMo.). Namely, results show a statistically significant increase (p<0.1) in the number of smooth muscle cells adherent on nanophase CoCrMo as compared to conventional CoCrMo.

### Longer Term RAEC and RASMC Growth on Titanium Substrates

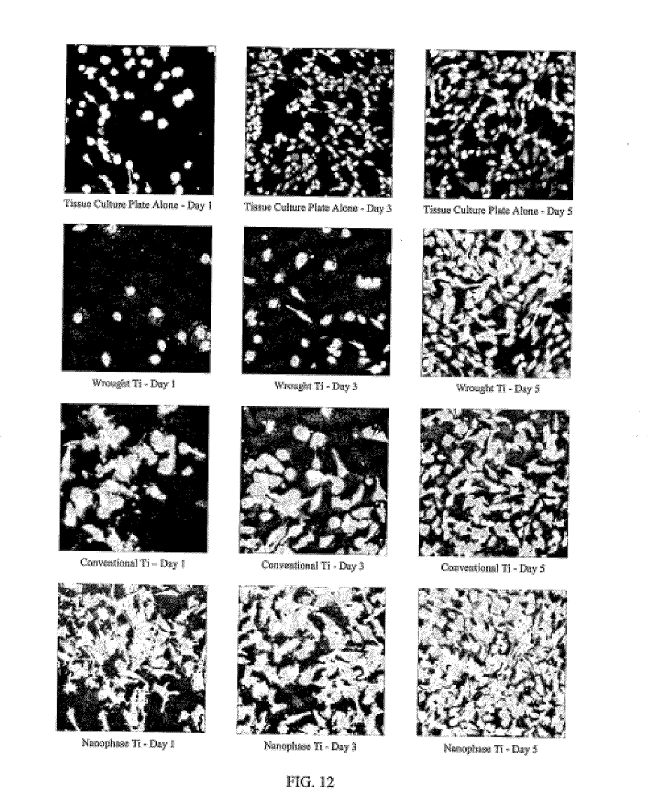

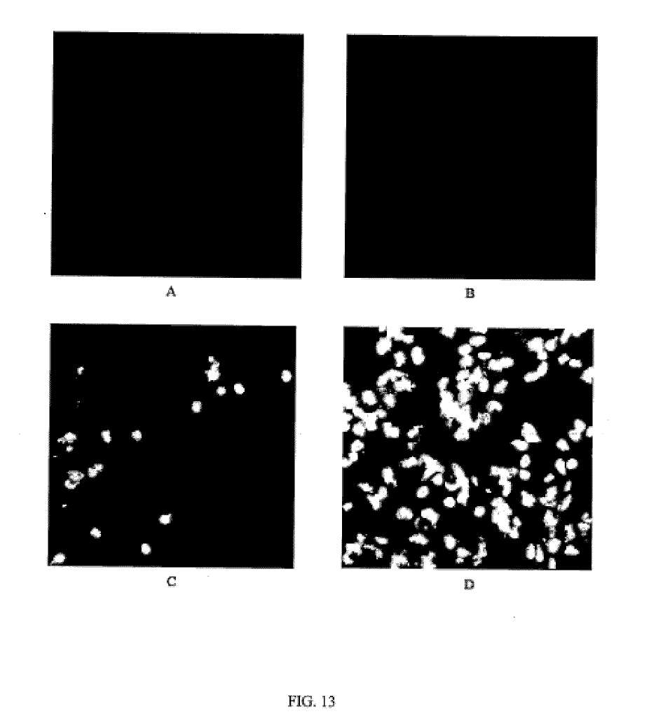

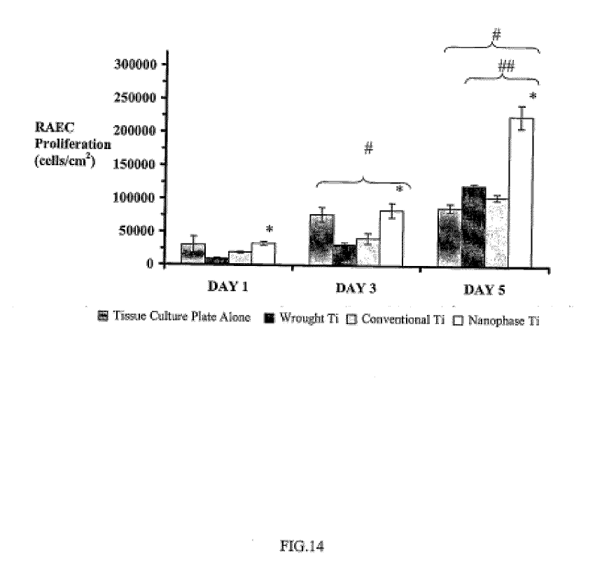

Adhesion is a first step when considering the acceptance of any biomaterial. However, for a biomaterial's long-term success, it is desirable that cells proliferate well on the implant surface. A series of experiments evaluating how well vascular cells grew (over time periods of 1, 3, and 5 days) on nanophase Ti compared to conventional and wrought Ti was undertaken, as show in FIG. 12. Qualitatively, FIG. 12 shows fluorescence microscopy images (magnification 20×) on which greater density of endothelial cells are present on nanophase titanium substrates compared to conventional or wrought titanium substrates on each of the days tested. In fact, a near-complete monolayer of endothelial cells can be seen on nanophase titanium by day 5 of culture. In addition, the cells on nanophase titanium displayed a more well-spread morphology as compared to endothelial cells present on each of the other substrates. After cell lysis, the substrates were stained using the live/dead assay (as described above) to confirm complete removal of endothelial cells. FIG. 13 shows images of each substrate after such lysis and staining. Images were taken under a fluorescence microscope at a magnification of 20×. Images are of dead endothelial cell remnants present after cell lysis on substrates from day 5. Similar results were seen on substrates from days 1 and 3 (not shown). In the figure, A: Tissue Culture Plate Alone—DAY 5; B: Wrought Ti—DAY 5; C: Conventional Ti—DAY 5; D: Nanophase Ti—DAY 5. It can be observed that endothelial cells on the plate alone and wrought Ti were completely detached; similarly, little to no cell remnants were found adherent to conventional Ti. However, a large number of cell remnants were found still adherent to nanophase Ti after cell lysis, suggesting a greater strength of adhesion of endothelial cells on nanophase Ti as compared to the other substrates tested.

Due to this finding and in order to avoid any resulting discrepancies in cell counts using these potentially “incomplete” cell lysates, a direct cell count (as described previously) was instead used to quantify the cell growth data. The graph in FIG. 14 shows the increased RAEC growth on nanophase titanium (data are mean values±SEM, *p<0.01 compared to conventional and wrought titanium at the same time point; #p<0.01 compared to respective day 1 substrates; ##p<0.01 compared to respective day 3 substrates). Using these direct cell counts, it was observed (FIG. 14) that the growth of endothelial cells was significantly greater (p<0.01) on nanophase titanium as compared to conventional and wrought titanium on all days tested. In addition, endothelial cells grew well over time on each of the substrates tested, which was evident by comparing the counts within one substrate on one day to the previous day. Specifically, there was a significantly higher (p<0.01) number of cells on days 3 and 5 on all substrates tested as compared to day 1 and there was a significantly higher (p<0.01) number of cells on day 5 on all metal substrates tested as compared to day 3.

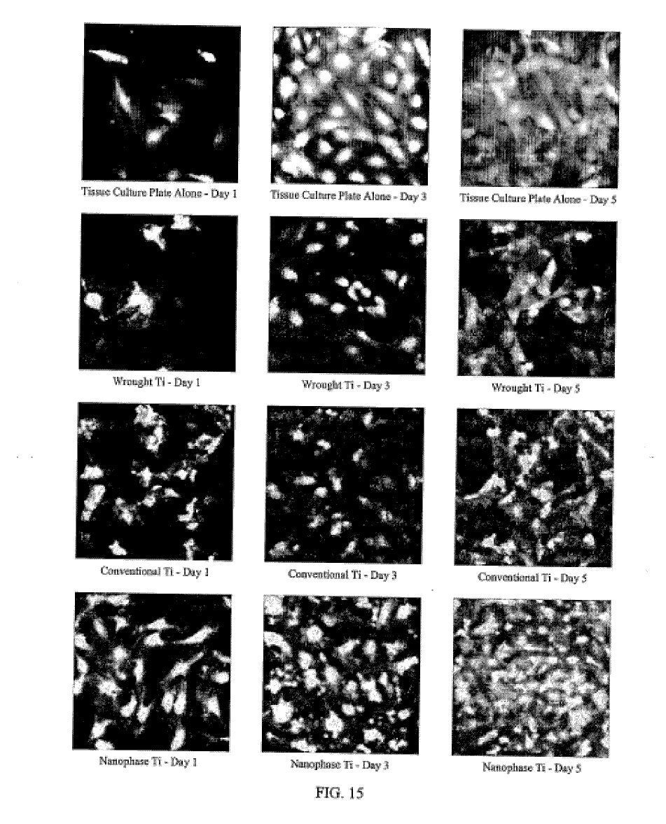

The representative images of live RASMC grown on substrates on day 1, day 3, and day 5 are shown in FIG. 15. Images were taken under a fluorescence microscope at a magnification of 20×. Qualitatively, FIG. 15 shows a greater density of vascular smooth muscle cells on nanophase Ti compared to conventional or wrought Ti on each of the days tested. In fact, smooth muscle cells grow to near-confluence on nanophase Ti by day 5 of culture. In addition, the cells on nanophase Ti displayed a more well-spread morphology as compared to smooth muscle cells present on each of the other substrates.

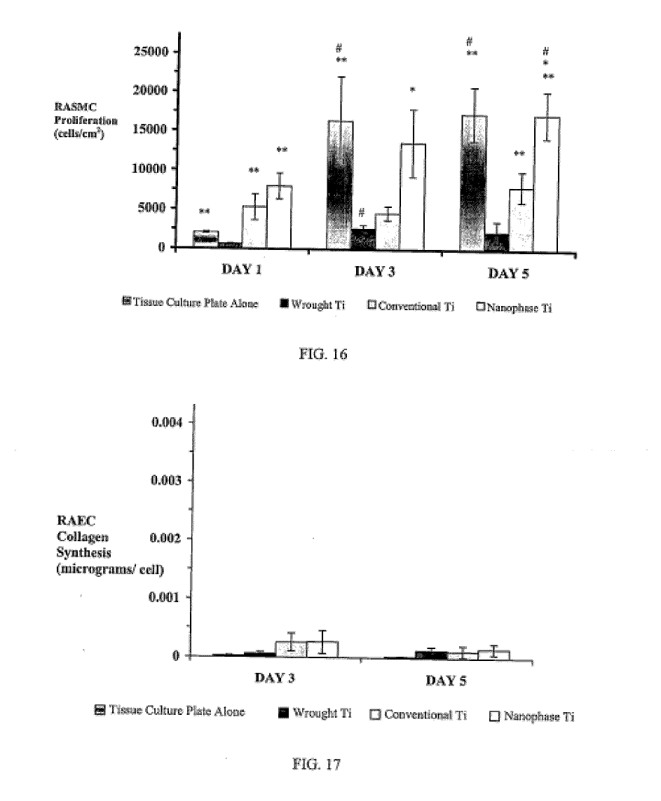

Similar to methods used with endothelial cells, vascular smooth muscle cells growing on each substrate were next lysed, stained, and viewed under a fluorescence microscope. In this case, little to no cells were found remaining on any of the substrates tested after 1, 3, and 5 days (data not shown). This suggests a weaker adhesion of smooth muscle cells to nanophase Ti. Therefore, the LDH assay (as previously described in Cell Count Assay section) was used to quantify cell count data at each time point and within each cell lysate. The graph in FIG. 16 shows increased RASMC growth on nanophase titanium (data are mean values±SEM; n=3; *p<0.1 compared to conventional titanium at the same time points **P<0.05 compared to wrought titanium at the same time point; #p<0.1 compared to respective day 1 substrate). Results suggested that the growth of vascular smooth muscle cells (FIG. 16) was significantly greater (p<0.1) on nanophase titanium compared to conventional titanium on days 3 and 5. There was also a significantly greater number of vascular smooth muscle cells present on nanophase titanium as compared to wrought titanium (p<0.05) on days 1 and 5. In most cases, the growth of smooth muscle cells was significantly greater (p<0.05) on conventional titanium as compared to wrought titanium. Finally, smooth muscle cells grew well on nanophase titanium over a course of 5 days, evident by the significantly higher (p<0.1) number of cells on day 5 as compared to day 1.

It was found that endothelial and vascular smooth muscle cells grew better on the substrate with nanostructured surface features (FIG. 14 and FIG. 16). In fact, by day 5 of culture, a near-complete monolayer of endothelial cells was formed on nanophase titanium (FIG. 12); vascular smooth muscle cells were also seen to grow to near-confluence on nanophase titanium (FIG. 15).

### Extracellular Matrix Component Synthesis

The synthesis of extracellular matrix (ECM) proteins was evaluated for cells growing on each substrate after 3 and 5 days. More specifically, the amount of collagen and elastin produced by each cell was measured (as described previously) in each cell lysate sample.

### Synthesis of Collagen by RAEC and RASMC

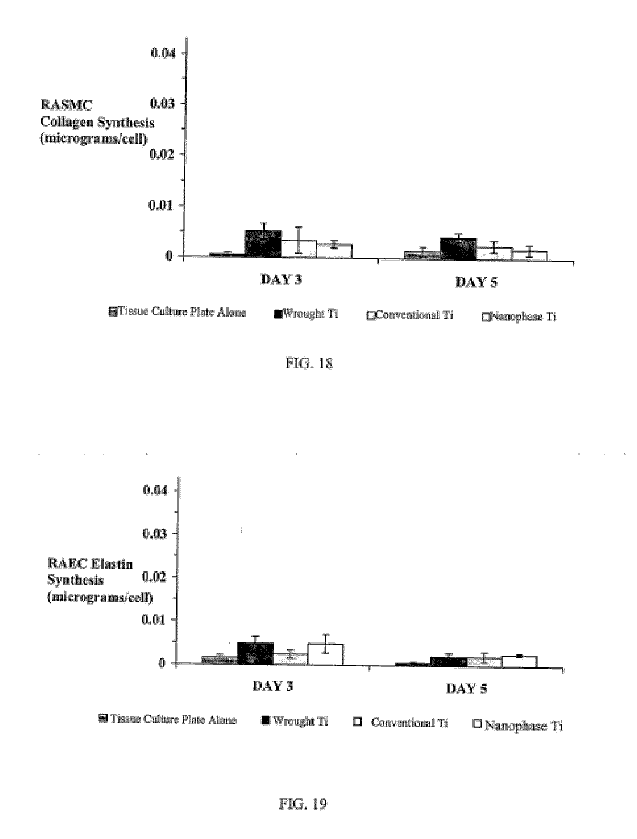

The amount of collagen produced by each individual endothelial and vascular smooth muscle cell, respectively, on the substrates of interest is shown in FIG. 17 (Collagen Synthesis Per RAEC was Similar on all Substrates. Data are mean values±SEM; n=3) and FIG. 18 (FIG. 18: Collagen Synthesis Per RASMC was Similar on all Substrates. Data are mean values±SEM; n=3). No significant differences were observed between the amount of collagen synthesized on a per cell basis comparing the different substrates within a single time point (e.g., day 3) and also comparing the same substrates between different time points (i.e., day 3 versus day 5). More collagen was produced by vascular smooth muscle cells than by endothelial cells. Since there was a greater number of endothelial cells present on nanophase titanium after 3 and 5 days, a greater total production of collagen was observed on nanophase titanium as compared to conventional and wrought titanium (data not shown).

### Synthesis of Elastin by RAEC and RASMC

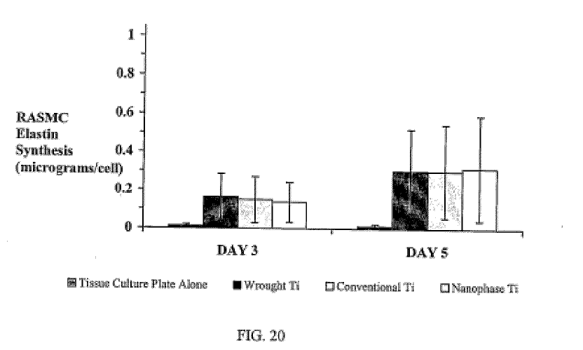

Evidence of a similar trend in elastin production by endothelial and vascular smooth muscle cells, is shown in FIG. 19 (FIG. 19: Elastin Synthesis Per RAEC was Similar on all Substrates. Data are mean values±SEM; n=3) and FIG. 20 (FIG. 20: Elastin Synthesis Per RASMC was Similar on all Substrates. Data are mean values±SEM; n=3), respectively. Specifically, the amount of elastin produced by each individual endothelial and vascular smooth muscle cell was the same regardless of the substrate or time point tested. As would be expected, more elastin was produced by smooth muscle cells than by endothelial cells. Since there was a greater number of smooth muscle cells present on nanophase titanium after 3 and 5 days, a greater total production of elastin was observed on nanophase Ti as compared to conventional and wrought titanium (data not shown). Finally, when comparing elastin and collagen production, individual endothelial and smooth muscle cells produced more elastin as compared to collagen.

Collagen and elastin production was maintained at a basal level for each substrate and cell type tested (FIG. 17-FIG. 20), indicating that the titanium substrates had no accelerating or hampering effects on the cells.

Note that the scanning electron microscopy images (FIG. 3 and FIG. 5) and AFM data (FIG. 1 and FIG. 2) results confirm that the amount of nanometer surface roughness was 2.4 and 1.9 times greater on nanophase titanium and CoCrMo compared to conventional titanium and CoCrMo substrates, respectively. This resulted in a 15% and 11% increase in surface area of nanophase titanium and CoCrMo Compared to conventional titanium and CoCrMo compacts, respectively.

Using these nanophase and conventional substrates, demonstrate that endothelial and vascular smooth muscle cells adhered in greater numbers (FIG. 6 and FIG. 8) and with a more well-spread morphology (FIG. 7 and FIG. 9) on nanophase titanium as compared to conventional or wrought titanium. This trend was also seen with the CoCrMo alloy (FIG. 10 and FIG. 11), showing that the preference of vascular cells to surfaces with nanostructured features holds for other metals and is not specific to titanium. In addition, nanophase titanium did not decrease the viability of the adherent endothelial and vascular smooth muscle cells. However, there was a slight decrease in the viability of endothelial cells on wrought titanium compared to all other substrates tested; this could have been due to the presence of contaminants from manufacturing and processing of the metal.

Vascular cells are shown to better accept the metal with nanostructured surface features, demonstrating that a nanophase stent could be incorporated into the endothelium much faster than a stent with conventional surface features.

In another embodiment, the regeneration of the endothelium lies in the use of biomaterials with nanostructured surface features. Namely, enhanced biocompatibility may be achieved by increasing the surface roughness and hence the surface area of vascular stents through the use of materials with biologically-inspired surfaces composed of nanometer grain sizes. This is because nanostructured surface features on an implanted material mimic the surface roughness of the natural host tissue; this familiar rough topography of the biomaterial enhances cellular activity.

While the invention has been illustrated and described in detail in the foregoing description, such an illustration and description is to be considered as exemplary and not restrictive in character, it being understood that only the illustrative embodiments have been described and that all changes and modifications that come within the spirit of the invention are desired to be protected.

1. A method of making an implantable composition, the method comprising forming an implantable composition consisting of a metallic component, said metallic component consisting of a metal selected from the group consisting of titanium, a titanium alloy, and CoCrMo, wherein said forming comprises providing the metal in the form of particles of nanophase powder, and compacting the particles, thereby forming the implantable composition, wherein the particles of nanophase powder have at least one dimension that is in the range of 1 to 2500 nm.

2. The method according to claim 1, wherein the implantable composition has a surface defined by a plurality of metallic structures, wherein said metallic structures are formed by particles of the nanophase powder present at an exterior surface of the implantable composition, the metallic structures being defined by a set of dimensions wherein at least one dimension of the set is equal to or less than 500 nm, wherein the at least one dimension is the height or width of the metallic structures.

3. The method according to claim 1, wherein a plurality of nanometer-sized voids having a diameter of less than 1 μm are distributed throughout the metallic component of the implantable composition.

4. The method according to claim 1, wherein a plurality of holes are formed in the metallic component.

5. The method according to claim 4, wherein the holes have an average diameter of less than 1 mm.

6. The method according to claim 5, wherein the holes have an average diameter of 100 to 800 μm.

7. The method according to claim 1, wherein a surface of the implantable composition exhibits a surface roughness of at least 11.9 nm.

8. The method according to claim 1, wherein the metal is titanium.

9. The method according to claim 1, wherein the metal is a titanium alloy.

10. The method according to claim 9, wherein the titanium alloy consists of titanium, aluminum, and vanadium.

11. The method according to claim 1, wherein the metal is CoCrMo.

12. The method according to claim 11, wherein the metal consists of 2-5 wt % cobalt, 60-80 wt % chromium, and 20-35 wt % molybdenum.

13. The method according to claim 1, wherein the implantable composition is a stent.

14. The method according to claim 1, wherein said compacting comprises pressing the particles.

15. The method according to claim 1, wherein said compacting comprises sintering the particles.

16. The method according to claim 1, wherein said compacting comprises using a laser.

17. The method according to claim 16, wherein said compacting comprises selective laser sintering.

18. An implantable composition consisting of a metallic component, said metallic component consisting of metal selected from the group consisting of titanium, a titanium alloy, and CoCrMo, wherein the metal is formed from particles of nanophase powder having at least one dimension that is in the range of 1 to 2500 nm.

19. The implantable composition according to claim 18, wherein the implantable composition has a surface defined by a plurality of metallic structures, wherein said metallic structures are formed by particles of the nanophase powder present at an exterior surface of the implantable composition, the metallic structures being defined by a set of dimensions wherein at least one dimension of the set is equal to or less than about 500 nm, wherein the at least one dimension is the height or width of the metallic structures.

20. The implantable composition according to claim 18, wherein a plurality of holes are present in the metallic component.
